(19) United States
(12) Patent Application Publication
(10) Pub. No.: US 2026/0192089 A1
(43) Pub. Date: Jul. 9, 2026

(54) CATHETER SYSTEM

(71) Applicant: Field Day, LLC, Waco, TX (US)

(72) Inventor: Adam Ylitalo, Waco, TX (US)

(21) Appl. No.: 19/255,292

(22) Filed: Jun. 30, 2025

Related U.S. Application Data
(60) Provisional application No. 63/743,330, filed on Jan. 9, 2025.

(51) Int. Cl. A61M 25/00; A61M 25/02; A61M 39/00; A61M 39/08

(52) U.S. Cl. CPC A61M 25/007; A61M 25/0026; A61M 39/08; A61M 25/0111; A61M 2025/0213; A61M 2039/0009

(57) ABSTRACT

A urinary catheter management system for allowing hands-free support of a catheter assembly to, in turn, allow unassisted bladder and urethra flushing, particular for urinary catheter users.

### CROSS-REFERENCE TO RELATED APPLICATION

This application is based on and claims the benefit of U.S. Provisional Application No. 63/743,330 filed on Jan. 9, 2025, the disclosure of which is incorporated by reference herein in its entirety.

### BACKGROUND

### 1. Field of the Invention

The present invention relates to catheters such as are primarily associated with urinary tract interventions and maintenance.

### 2. Background of the Invention

A urinary catheter is a medical device used to drain urine from the bladder when an individual is unable to do so independently or effectively. The need for urinary catheters arises in various medical situations, including temporary and long-term conditions, where individuals cannot fully empty their bladder due to injury, illness, or surgical procedures.

Urinary catheters are vital medical devices used to address a wide variety of conditions affecting bladder function. They provide a necessary solution for individuals unable to manage urinary function independently due to medical or neurological conditions, surgeries, or injuries. Currently, catheter use must be carefully monitored to minimize risks and complications, and ongoing care is required to optimally maintain hygiene and prevent infections.

Use of urinary catheters can be either short-term or long-term. Short-term use is often for use during hospital stays for monitoring urine output post-surgery or in critical care settings where immobile patients cannot access toilet facilities. Long-term use involves addressing chronic retention or incontinence. In cases where patients are unable to void naturally due to significant nerve damage, disease, or advanced age-related conditions (BPH in males, for example), long-term catheterization use can provide necessary management of urine flow.

Patients who are able to do so may perform intermittent catheterization, where the catheter is inserted and removed multiple times a day to drain urine when they cannot urinate normally. This method can help avoid long-term catheter-related complications, but certain risks to patients remain in the context of current practices and available devices.

One of the most common and serious complications associated with catheter use is urinary tract infections (UTIs). The presence of a catheter introduces a potential entry point for bacteria into the urinary system. Over time, bacteria can build up within the catheter or bladder, leading to infection.

Regular urethral and bladder flushing is a necessary, but often omitted step in helping clear any bacterial buildup, debris, and sediment that may accumulate, and thus significantly reduce the risk of infection. By flushing out the bladder with sterile or prescribed fluids, bacteria and pathogens are removed, or at least materially reduced before they have a chance to colonize and cause infection. However, this is not a procedure that, in the current state of medical devices and practices, patients can readily perform themselves.

The optimal regimen for urethral and bladder flushing depends on the type of catheter used, the patient's medical condition, as determined by the involved healthcare providers. In some cases, flushing may need to be done daily or more frequently, particularly if there are signs of infection or catheter blockage. Clearly, if a patient is unable to perform the flushing procedure independently, significant costs and inconveniences are implicated through the then-necessary involvement of third party assistance, likely in a medical facility or home healthcare context.

In view of the above, facilitating a urinary catheter user to independently flush their bladders and urethras, as opposed to omitting this important process or relying on healthcare professionals, can offer several significant benefits. These advantages touch on convenience, autonomy, cost-effectiveness, health outcomes, and patient satisfaction. In addition to the costs and time savings that are apparent from the above observations, a more acute benefit for self-flushing or irrigation will be available—that of flushing in the event of an acute blockage without the remediation of which, most seriously, kidney damage may result. This, in addition, to the considerable discomfort and associated complications that can arise in such instances.

Further still, the ability to manage one's own catheter care can have psychological benefits. Having the ability to care for oneself, especially in a condition that may be stigmatized or uncomfortable for some, can boost confidence and reduce feelings of helplessness. It helps reinforce a sense of control and agency, which is critical for mental well-being. Moreover, this sense of mastery can lead to improved overall satisfaction with one's healthcare regimen and a greater sense of empowerment.

In view of the foregoing, the present inventor here discloses a system that, in totality, or even through use of certain components facilitates the autonomous bladder and urethra flushing as is most typically, though not exclusively needed by urinary catheter users.

The present inventor's system includes, in a preferred embodiment, a catheter assembly with an insertion segment that is to be inserted through a user's urethra with effluent ports for discharge of flushing fluid(s) into the user's bladder, as well as an evacuation port for accepting flow from the bladder. The insertion segment is, in a preferred embodiment, a single lumen conduit that is in sealed fluid communication with two conduits, an inflow conduit and an outflow conduit. The inflow conduit is for accepting flushing fluid(s), such as saline, while the outflow conduit will discharge evacuated bladder and fluids to a suitable receptacle. Of course, fluid introduced into the inflow conduit, passing through the insertion segment, and existing from the catheter assembly through effluent port(s) will, in anticipated operation, reenter the catheter assembly's second lumen through the evacuation port to be discarded through the outflow conduit.

The system in a preferred embodiment further includes a support structure that engages with the catheter assembly in such a way that is supported, hands-free, in an ideal position for use, thus freeing the user's hands to perform the insertion, withdrawal and other steps in the flushing or irrigation process.

The system and its embodiments here disclosed facilitate a user's optimal urinary tract maintenance as relates to urinary catheter use, in a cost-effective, convenient and autonomous manner not presently possible with currently known or available devices or methods.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 is an elevational view representation of the disclosed catheter assembly portion of the present system.

FIG. 2 is an elevational view representation of the disclosed catheter assembly support frame and the disclosed catheter assembly support interface.

FIG. 3 is an elevational view representation of an alternative embodiment of the disclosed catheter assembly portion of the present system.

FIG. 4 is a cross sectional view of insertion segment 12 of the present system, along line A-A.

### DETAILED DESCRIPTION OF THE PREFERRED EMBODIMENT

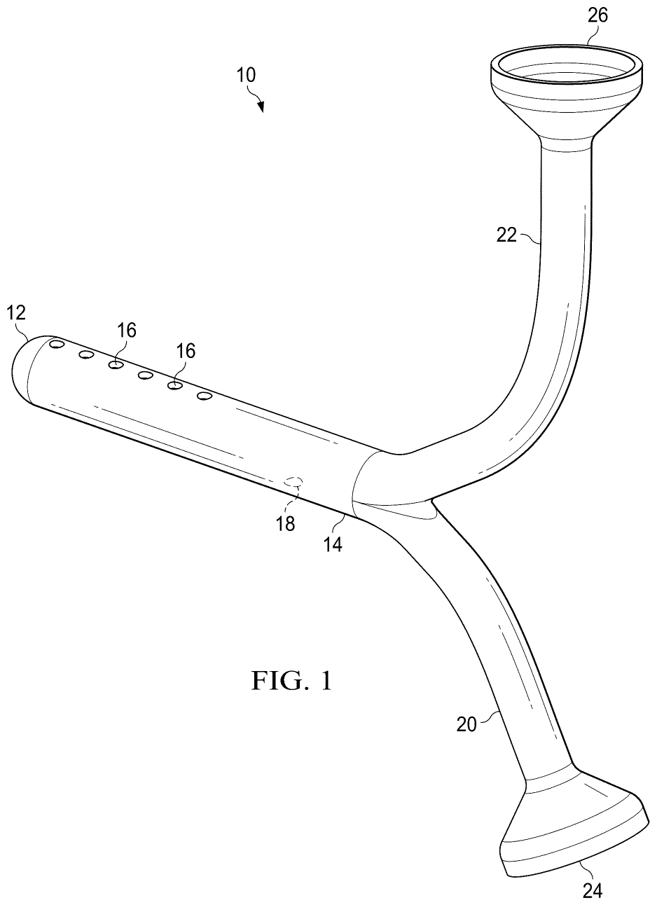

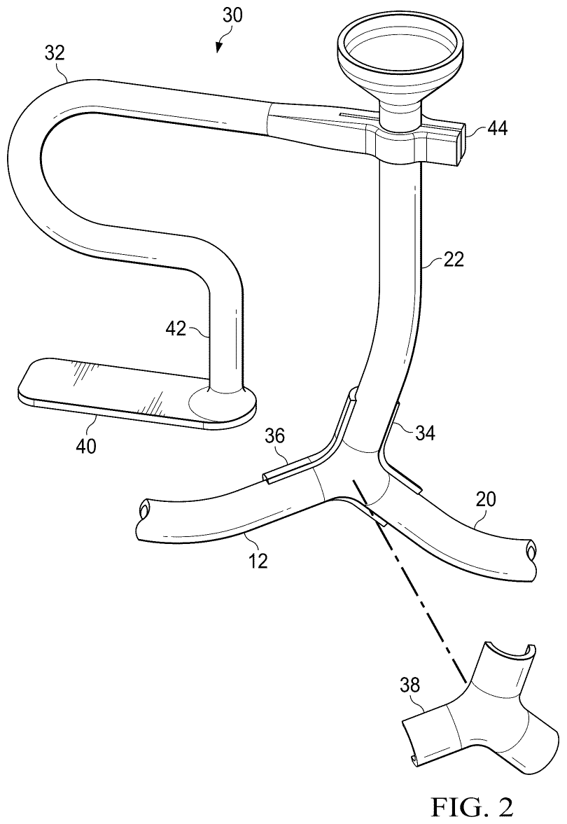

FIGS. 1 and 2, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged system or device.

Referring primarily to FIG. 1, a preferred embodiment of the present inventor's catheter system includes a catheter assembly referenced generally in FIG. 1 by the numeral 10. Assembly 10 includes an insertion segment 12 that, when used, is to be inserted through a user's urethra.

Insertion segment 12 is, in a preferred embodiment a conduit that has effluent port(s) 16 formed through a portion of the outer surface near the proximal, insertion end of the insertion segment 12 (proximal, relative to the user). Extending from and in fluid communication with insertion segment 12 is an inflow conduit 22. Attached to the insertion segment is an outflow conduit 20. The inflow conduit 22 is for accepting and directing into the insertion segment 12 flushing fluid(s), such as saline that are to be introduced into a user's bladder for flushing purposes.

Outflow conduit 20 is at least substantially isolated from direct fluid communication with inflow conduit 22. Outflow conduit 20 includes evacuation port(s) 18 at or near a proximal end 14 (proximal relative to the user) of, and opening into outflow conduit 20 to provide passage fluids from a user's bladder (likely, mostly the flushing fluid introduced via the inflow conduit 22 and insertion segment) into the interior lumen of the outflow conduit 20 to be discharged from system 10 at a distal end 24 (distal, relative to the user) of outflow conduit 20 (or from any extension, addition or attachment thereto).

Outflow conduit 20 is positioned relative to insertion segment 12 such that evacuation port(s) 18 will reside within, or at least effectively positioned relative to a user's bladder opening to receive post-flushing fluids that have previously been introduced into the bladder via inflow conduit 22.

In short, fluid introduced into the inflow conduit 22, passing through the insertion segment 12, and exiting from the catheter assembly through evacuation port(s) 18 will, in anticipated operation, reenter the catheter system's outflow conduit 20 for routing to a further disposal circuit or receptacle (not shown in the drawings).

Whether insertion segment 12, conduits 20 and 22 unitarily molded structures, or are wholly or partially separate/separable components that are assembled for final use may be the product of a design and manufacturing choice. However, it should be noted that having at separable components would allow, for example, assemblage of systems with differing sizes of sub-components, as opposed to having unitary structures for each of any multiple desired parameters. For example, male and female users will ideally use insertion segments of differing lengths to accommodate their respective anatomy, and have a modular assembly of separable subcomponents would allow for the assembly of the ultimately desired system from separately available such components. In such a system, suitable junction means between individual components would be included (not shown in the drawings, but well within the skills of persons of ordinary skill in the art to design and fabricate).

As will be apparent to appropriate practitioners, insertion segment 12 will be available in suitable lengths to accommodate, for example, use with male and female patients and is, in a preferred embodiment, a single conduit from which the below-described conduits 22 and 24 extend and are in fluid communication.

Referring to FIG. 2, the system in a preferred embodiment further includes a support assembly 30 that engages with the catheter assembly in such a way that is supported, hands-free, in an ideal position for use, thus freeing the user's hands to perform the insertion, withdrawal and other steps in the flushing or irrigation process.

Support assembly 30 includes a frame 32 and a catheter interface 34. Interface 34 is configured to both attach to frame 32 and to engage with catheter assembly 10 (depicted in dashed lines in FIG. 20). One embodiment of the support assembly includes an interface 34 that comprises halves 36 and 38, one of which is envisioned to be permanently attached to frame 32, while the other is reversibly engageable in a snap-lock configuration to allow engagement and disengagement of the catheter assembly 10 to and from the frame 32. The particular mode of attachment of interface 34 with frame 32, or the reversible engagement with catheter assembly 10 is not material to practice of the present invention, so long as the catheter assembly is supported and positioned for use by frame 32 once positioned by a user.

Frame 32 includes several integral or joined segments that, in cooperation, provide for anchoring by a user's leg (anchor segment 40), circumventing and engaging the user's leg (leg circumvention segment 42), and a positioning segment 44 extending from the circumvention segment 42 and, when so anchored and engaged with a user's leg, spatially positioning the attached interface 34 (with the attached catheter assembly 10) relative to the user for facilitating the referenced hands-free support and use of catheter assembly 10. The referenced segments 40, 42 and 44 may be general, ill-defined regions of a unitary structure, rather than distinctly delineable components. On the other hand, frame 32 may be configured of separate, somewhat customizable components that may be selected according to differing parameters such as a patient's thigh dimensions, the desired positioning of the frame-supported catheter assembly relative to the patient with the frame 32 is supported as described, and so forth.

An alternative embodiment of the frame of the support assembly (not shown in the drawings) may omit the “leg circumvention segment” of the above-described embodiment, and instead include only a segment corresponding to the “anchor segment” mentioned above configured for either a patient to simply rest a leg upon, wedge between a toilet seat and toilet rim, for example. In such a configuration, the segment corresponding to the “positioning segment” as described above would be sized and contoured to position this version's catheter interface in a desired position for use.

The system and its embodiments here disclosed facilitate a user's optimal urinary tract maintenance as relates to urinary catheter use, in a cost-effective, convenient and autonomous manner not presently possible with currently known or available devices or methods.

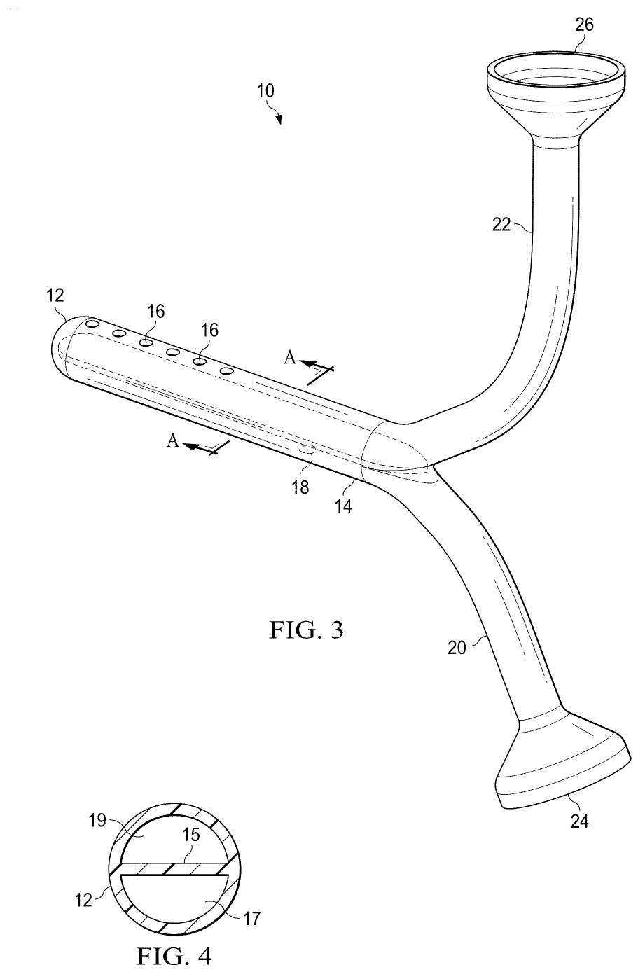

Referring to FIGS. 3 and 4, an alternative embodiment of the present system may involve an insertion segment 12 that is a dual-lumen conduit, with a septum 15 (not visible from the outside of insertion segment 12, but represented by line 13 in FIG. 3). Septum 15 divides and separates lumens 17 and 19. Into each of lumens 17 and 19 respectively open effluent port(s) 16 and evacuation port(s) 18. Lumens 17 and 19 are respectively in sealed fluid communication with into inflow conduit 22 and outflow conduit 20.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

Although the present disclosure has been described with various embodiments, various changes and modifications may be suggested to one skilled in the art. It is intended that the present disclosure encompass such changes and modifications as fall within the scope of the appended claims.

1. A catheter system comprising:
a catheter assembly comprising an insertion segment conduit, an inflow conduit and an outflow conduit, said insertion segment conduit being in substantially sealed fluid communication with a proximal ends of said inflow conduit;
said insertion segment conduit having a plurality of effluent ports through which flushing fluids introduced into said catheter assembly through said inflow conduit may exit said catheter assembly from said insertion segment conduit to flush a bladder of a user from the plurality of effluent ports;
said outflow conduit having an evacuation port positioned substantially at a proximal end of said outflow conduit for receiving flushing fluid and urine exterior of said catheter assembly within the bladder of the user for evacuation through said outflow conduit at a distal end of said outflow conduit.

2. The system of claim 1 further comprising:
a catheter support frame; and
a catheter interface positioned on said catheter support frame and configured for reversible engagement with said catheter assembly.

3. The system of claim 2 wherein said catheter support frame is configured to include a leg circumvention segment and a positioning segment.

4. The system of claim 2 wherein said catheter support frame is configured to include an anchor leg segment and a positioning segment.

5. The system of claim 1 wherein:
said insertion segment is reversibly engageable with said inflow conduit with said insertion segment and said inflow conduit being mutually configured for transition between disengagement and substantially sealed fluid communication with each other; and
wherein said insertion segment is reversibly engageable with said outflow conduit with said insertion segment and said outflow conduit being mutually configured for transition between disengagement and substantially sealed fluid communication with each other.

6. The system of claim 1 wherein said insertion segment is reversibly engageable with said inflow conduit with said insertion segment and said inflow conduit being mutually configured for transition between disengagement and substantially sealed fluid communication with each other.

7. The system of claim 1 wherein said insertion segment is reversibly engageable with said outflow conduit with said insertion segment and said outflow conduit being mutually configured for transition between disengagement and substantially sealed fluid communication with each other.

8. The system of claim 2 wherein:
said insertion segment is reversibly engageable with said inflow conduit with said insertion segment and said inflow conduit being mutually configured for transition between disengagement and substantially sealed fluid communication with each other; and
wherein said insertion segment is reversibly engageable with said outflow conduit with said insertion segment and said outflow conduit being mutually configured for transition between disengagement and substantially sealed fluid communication with each other.

9. The system of claim 2 wherein said insertion segment is reversibly engageable with said inflow conduit with said insertion segment and said inflow conduit being mutually configured for transition between disengagement and substantially sealed fluid communication with each other.

10. The system of claim 2 wherein said insertion segment is reversibly engageable with said outflow conduit with said insertion segment and said outflow conduit being mutually configured for transition between disengagement and substantially sealed fluid communication with each other.

11. The system of claim 3 wherein:
said insertion segment is reversibly engageable with said inflow conduit with said insertion segment and said inflow conduit being mutually configured for transition between disengagement and substantially sealed fluid communication with each other; and
wherein said insertion segment is reversibly engageable with said outflow conduit with said insertion segment and said outflow conduit being mutually configured for transition between disengagement and substantially sealed fluid communication with each other.

12. The system of claim 3 wherein said insertion segment is reversibly engageable with said inflow conduit with said insertion segment and said inflow conduit being mutually configured for transition between disengagement and substantially sealed fluid communication with each other.

13. The system of claim 3 wherein said insertion segment is reversibly engageable with said outflow conduit with said insertion segment and said outflow conduit being mutually configured for transition between disengagement and substantially sealed fluid communication with each other.

14. The system of claim 4 wherein:
said insertion segment is reversibly engageable with said inflow conduit with said insertion segment and said inflow conduit being mutually configured for transition between disengagement and substantially sealed fluid communication with each other; and
wherein said insertion segment is reversibly engageable with said outflow conduit with said insertion segment and said outflow conduit being mutually configured for transition between disengagement and substantially sealed fluid communication with each other.

15. The system of claim 4 wherein said insertion segment is reversibly engageable with said inflow conduit with said insertion segment and said inflow conduit being mutually configured for transition between disengagement and substantially sealed fluid communication with each other.

16. The system of claim 4 wherein said insertion segment is reversibly engageable with said outflow conduit with said insertion segment and said outflow conduit being mutually configured for transition between disengagement and substantially sealed fluid communication with each other.

17. A method for irrigating a patient's bladder comprising the steps of:
selecting a catheter assembly, said catheter assembly comprising
a catheter assembly comprising an insertion segment conduit, an inflow conduit and an outflow conduit, said insertion segment conduit being in substantially sealed fluid communication with a proximal ends of said inflow conduit;
said insertion segment conduit having a plurality of effluent ports through which flushing fluids introduced into said catheter assembly through said inflow conduit may exit said catheter assembly from said insertion segment conduit to flush a user's bladder from the plurality of effluent ports;
said outflow conduit having an evacuation port positioned substantially at a proximal end of said outflow conduit for receiving flushing fluid and urine exterior of said catheter assembly within the bladder of the user for evacuation through said outflow conduit at a distal end of said outflow conduit;
inserting said insertion segment through the user's urethra and for positioning said plurality of effluent ports and said evacuation port substantially within the user's bladder; and
introducing the flushing fluid through said inflow conduit to flush the user's bladder from the plurality of effluent ports.

18. The method of claim 17 wherein said system further comprises:
a catheter support frame; and
a catheter interface positioned on said catheter support frame and configured for reversible engagement with said catheter assembly; and further comprising the steps of:
engaging said catheter assembly with said catheter interface; and
supporting said catheter support frame whereby said catheter assembly is supported in substantial proximity to a user's urethral opening.

19. The system of claim 18 wherein said catheter support frame is configured to include a leg circumvention segment and a positioning segment.

20. The system of claim 18 wherein said catheter support frame is configured to include an anchor leg segment and a positioning segment.
